(19) United States
(12) Patent Application Publication
(10) Pub. No.: US 2010/0267076 A1
(43) Pub. Date: Oct. 21, 2010

(54) Separation of Co-Cultivated Cell Populations

(76) Inventor: Ute Schäfer, Gummersbach (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/680,956

(22) PCT Filed: Sep. 30, 2008
(86) PCT No.: PCT/EP08/63079

§ 371 Date: Jun. 25, 2010

(30) Foreign Application Priority Data
Oct. 2, 2007 (DE) 10 2007 047 321.6

(51) Int. Cl. C12Q 1/02

(52) U.S. Cl. 435/29

(57) ABSTRACT

The invention relates to a method and the use of an apparatus for separating co-cultivated cell populations.

The invention relates to the use of an apparatus as well as a method for separating co-cultivated cell populations.

Co-cultivation is possible for various biological systems (types of tissue/cells). In particular, animal and human embryonic stem cells are often primarily cultivated on a feeder cell monolayer, wherein the feeder cells provide growth factors.

The main interest of embryonic stern cell research focuses on the differentiation into specialized cells, in order to make these available for possible cell replacement therapies. For culturing stem cells, for example feeder cells are used. Cells of an embryo are dissociated and cultivated as a monolayer. The heterogeneous cell population may be deactivated by irradiation or mitomycin treatment, so that the cells cannot divide any longer. The feeder cells, however, are still metabolically active. Proliferation of embryonic stem cells depends on substances released into the medium by feeder cells. In order to differentiate stem cells, the overall culture, including the feeder cells, is removed from the cultivation plate used. In suspension cultures, the stem cells first may develop into embryoid bodies in the so-called hanging drops. These embryoid bodies are a mixed culture of stem cells and feeder cells. In the next differentiation steps, too, during which the embryoid bodies are differentiated into different cell types by addition of specific messengers, the cultures are contaminated by feeder cells. Since the stem cells divide in the early differentiation phases, the feeder cells, however, were deactivated, the quantitative ratio of the stem cell and feeder cell portion changes. In case of co-cultivation with feeder cells, the stem cells are thus not present in a pure state, but mixed with feeder cells. The feeder cell contamination may also be verified to a low extent in differentiated cell populations. This represents a problem above all for regenerative cell replacement therapies, since together with the stem cells, the feeder cells are transplanted into respective organs. The effect of the heterogeneous feeder cell population in the transplanted organ is not described. However, it could be demonstrated in examinations, that the feeder cells modulate the function of differentiated stem cells and that transplanted feeder cells, even weeks after transplantation, still could be verified in the respective organ. For this reason, complete separation of stem cells and feeder cells at an early stage is indispensable.

Thus, the object of the invention is the separation of co-cultivated cell populations.

The object is solved by using an apparatus for separating at least two co-cultivated cell populations with the characteristics of claim 1 and using a method for separating at least two co-cultivated cell populations with the characteristics of claim 5. The sub-claims contain suitable or advantageous, respectively, embodiments and characteristics of the method or use, respectively.

According to the invention, an apparatus is used for the separation of at least two co-cultivated cell populations, which comprises a microscope unit (1) for microscopic scanning of the cell culture (8), which comprises at least two co-cultivated cell populations, in combination with an imaging unit (2) and an image evaluation unit (3) for position detection of the cells in the cell culture (8), a control and storage unit (4) for storing the detected position of the cells, and a harvesting module (5) with a removal tool (10a) for removing the cells at the detected position of the cell.

The apparatus used according to the invention is described in detail in the international application PCT/EP2007/059951. Any technical characteristics disclosed in PCT/EP2007/059951 are included in the teaching of the present invention.

Within the scope of separation of various co-cultivated cell populations, diverse combinations of cell populations are possible. In particular, at least two co-cultivated cell populations may be separated, which comprise feeder cell and stem cell populations. Since the aspiration conditions may be adapted to the cultivation conditions, the method may also be applied to the separation of other co-cultivated cell populations. Further embodiments concern the use for separation of endothelial cell populations and populations of cells of the smooth muscles as well as for separation of endothelial cell and liver cell populations.

It could be demonstrated that a reproducible and complete separation of feeder and stem cells can be achieved, and that this method of stem cell purification does not have any influence whatsoever on the pluripotency of the stem cells.

The method according to the invention for separating at least two co-cultivated cell populations comprises the execution of a first detection step for selecting cells of a first cell population on the basis of material and/or physical parameters and recording of position data and storing the recorded position data of the selected cells in a position. database. The method is characterized by the following process steps:
execution of at least one second detection step for detecting at least one further parameter of the cells of the first cell population,
generation of comparative data from the data of the first and second detection step and allocation of the comparative data to the position data,
selection of cells on the basis of the comparative data, and
transmission of the position data linked with the comparative data from the position database to a harvesting unit.

The method corresponds to the method described in the international application PCT/EP2007/059951. Any particularities of the method disclosed in PCT/EP2007/059951 are included in the teaching of the present invention.

In a preferred embodiment, the method for separating at least two co-cultivated cell populations is characterized in that the at least two co-cultivated cell populations to be separated comprise feeder cell and stem cell populations. It is likewise preferred to use the method for separating endothelial cell populations and populations of cells of the smooth muscles as well as for separating endothelial cell and liver cell populations.

For cultivating stem cells, feeder cells are for example obtained from 13.5-day-old mouse embryos. Organs and head are removed from the embryos. The cells of the remaining embryo are dissociated and cultivated as a monolayer. The heterogeneous cell population is deactivated by irradiation or mitomycin treatment, so that the cells cannot divide any longer, wherein the feeder cells, however, remain metabolically active. The method according to the invention for separating at least two co-cultivated cell populations comprises the selection of cells of a first cell population on the basis of material and/or physical parameters. As can be detected using light microscopy, for example, feeder cells and stem cells have different structures or shapes, respectively (comp. FIG. 1). Under light microscopy it can be clearly detected, that stem cells grow as almost circular colonies on the monolayer of the elongated feeder cells. Accordingly, using the apparatus of PCT/EP2007/059951, the separation of the various cell populations is surprisingly possible.

In the following, the use of the apparatus according to PCT/EP2007/059951 as well as the method are to be set forth in more detail on the basis of embodiments.

### EXAMPLES

### Example 1

### Separation of Stem and Feeder Cells

Stem cells (D3) were cultivated on neomycin-resistant feeder cells for 5-8 days. Stem cells of individual colonies were aspirated with the tool for non-floating cells (glass capillary) under standardized conditions (suction pressure, amount of liquid). Using the apparatus according to PCT/EP2007/059951, it was possible to individually transport stem cells picked from various colonies within a Petri dish into specific wells, so that individual clones could be examined. The feeder cell monolayer was transfected with the neomycin resistance gene. This gene is not expressed in the stem cell line D3. Following successful separation of the cell populations, it should respectively not be possible to verify the neomycin resistance gene in the aspirated stem cell clones. The separation of stem and feeder cells is shown in FIGS. 2A to 2E. These figures show the repeated aspiration of stem cells of one colony (microscopic analysis). An expression analysis (RT-PCR) of the neomycin gene expressed in feeder cells is shown in FIG. 3. The microscopic analysis and the subsequent expression analysis clarify, that the aspiration of the stem cells could be repeated several times, before the feeder cells of the monolayer were co-aspirated. Only after the fifth repetition, feeder cells were aspirated as well. The verification took place on the basis of the expression of the neomycin gene in track E of the electrophoretically separated agarose gel.

### Example 2

### Verification of the Functional Integrity of the Stem Cells Following Purification (Separation from Feeder Cells)

Embryonic stem cells are pluripotent, i.e. in vitro they may be differentiated into different cell types. In order to verify that the early separation of the embryonic stem cells from the feeder cells does not attenuate the ability of the stem cells for differentiation into specific cell types, the differentiation potential of feeder-free stem cells and stem cells cultivated according to the standard method, i.e. with feeder cells, was compared on the basis of neuronal differentiation. The neuronal differentiation was analyzed on the basis of morphological criteria, but also on the basis of the expression of markers expressed in the various phases of neuronal differentiation. At no time, significant differences could be found in the differentiation potential of the feeder-free stem cells and the stem cells differentiated according to the standard protocol (comp. FIG. 3). It could be demonstrated, that a reproducible and complete separation of feeder and stem cells can be achieved and that this method of stem cell purification does not have any influence whatsoever on the pluripotency of the stem cells (comp. FIG. 4).

### DESCRIPTION OF THE FIGURES

FIG. 1 Light microscope image of stem and feeder cells (stem cells cultivated on feeder cells)

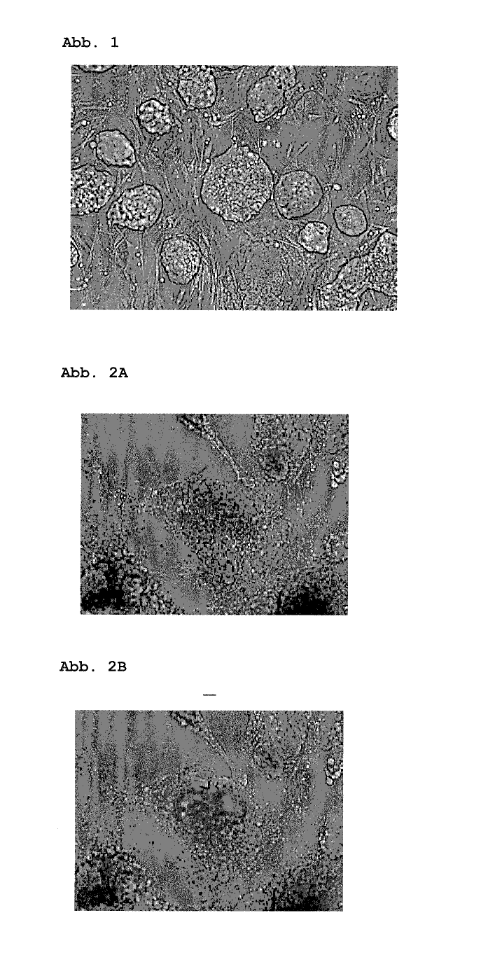

FIG. 2A to 2E: Repeated aspiration of stem cells of one colony (microscopic analysis)

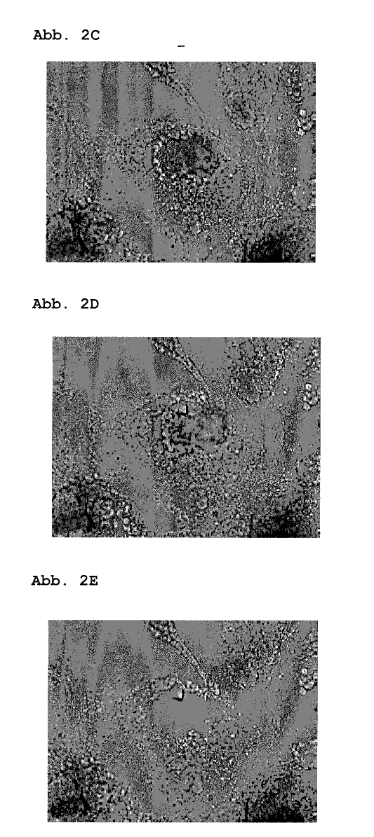

FIG. 3: Expression analysis (RT-PCR) of the neomycin gene expressed in feeder cells

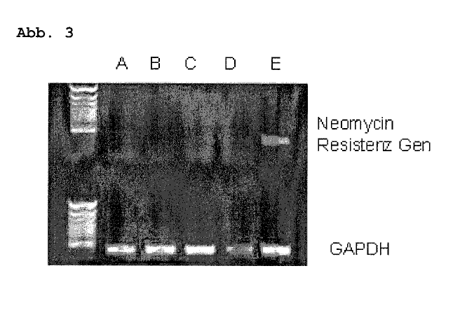

FIG. 4: Verification of the differentiation potential (comparison feeder-free stem cells and stem cells differentiated according to standard protocol)

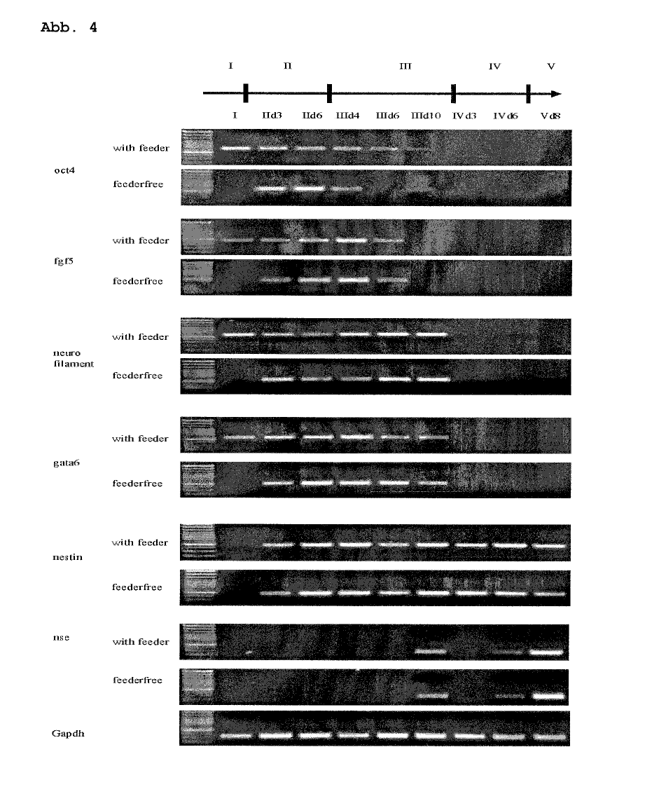

FIG. 5: Apparatus according to PCT/EP2007/059951 in an exemplary embodiment

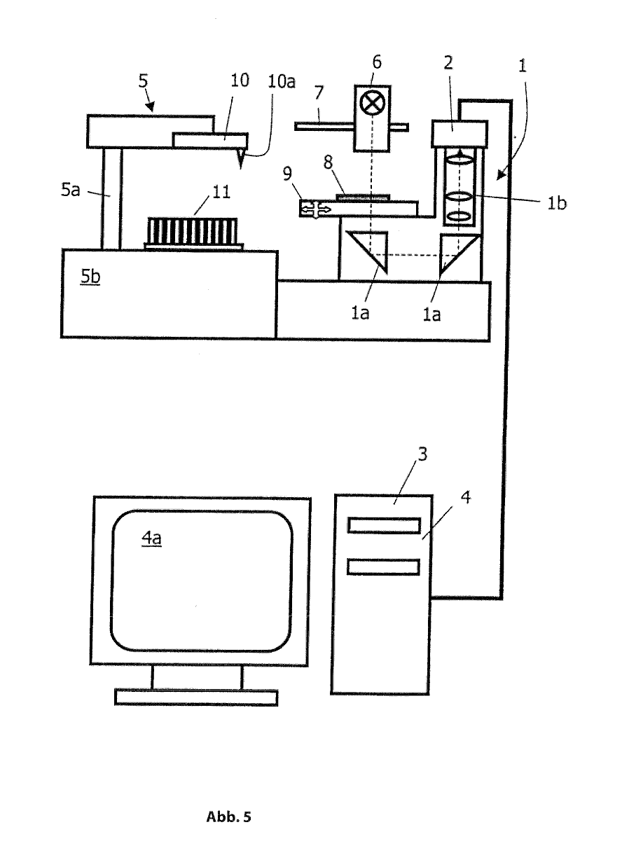

### LIST OF REFERENCE NUMBERS

1 Microscope unit
Ia Deflecting prism
Ib Lens system
2 Imaging unit
3 PC
3a Image evaluation unit
4 Control and storage unit
4a Monitor, display
5 Harvesting module
5a Lifting column
5b Traverse drive
6 Illumination
7 Illumination filter
8 Cell culture
9 xy table
10 Tool head
10a Removal tool
11 Separating battery

1. Use of an apparatus, comprising a microscope unit (1) for microscopic scanning of the cell culture (8), which comprises at least two co-cultivated cell populations, in combination with an imaging unit (2) and an image evaluation unit (3) for position detection of the cells in the cell culture (8), a control and storage unit (4) for storing the detected position of the cells, and a harvesting module (5) with a removal tool (10a) for removing the cells at the detected position of the cell, for separating at least two co-cultivated cell populations.

2. The use according to claim 1, characterized in that said at least two co-cultivated cell populations to be separated comprise feeder cell and stem cell populations.

3. The use according to claim 1, characterized in that said at least two co-cultivated cell populations to be separated comprise endothelial cell populations and populations of cells of the smooth muscles.

4. The use according to claim 1, characterized in that said at least two co-cultivated cell populations to be separated comprise endothelial cell and liver cell populations.

5. A method for separating at least two co-cultivated cell populations under
execution of a first detection step for selecting cells of a first cell population on the basis of the material and/or physical parameters surface area, size and/or outline and/or the spectral parameters brightness and/or fluorescence intensity, and
recording of position data and storing of the recorded position data of the selected cells in a position database,
characterized by the following process steps:
execution of at least one second detection step for detecting at least one further parameter of the cells of the first cell population only in those areas, in which material of interest was found during the first detection step,
generation of comparative data from the data of the first and second detection step and allocation of the comparative data to the position data,
selection of cells with several specific characteristics on the basis of the comparative data with the material and/or physical parameters and/or spectral parameters and on the basis of specific criteria for different cell types, and
transmission of the position data linked with the comparative data from the position database to a harvesting unit.

6. The method for separating at least two co-cultivated cell populations according to claim 5, characterized in that said at least two co-cultivated cell populations to be separated comprise feeder cell and stem cell populations.

7. The method for separating at least two co-cultivated cell populations according to claim 5, characterized in that said at least two co-cultivated cell populations to be separated comprise endothelial cell populations and populations of cells of the smooth muscles.

8. The method for separating at least two co-cultivated cell populations according to claim 5, characterized in that said at least two co-cultivated cell populations to be separated comprise endothelial cell and liver cell populations.
